(19) United States
(12) Patent Application Publication
(10) Pub. No.: US 2026/0157641 A1
(43) Pub. Date: Jun. 11, 2026

(54) BIOLOGICAL INFORMATION MEASUREMENT DEVICE

(71) Applicant: OMRON HEALTHCARE Co., Ltd., Kyoto (JP)

(72) Inventors: Shuhei MUNEHIRA, Kyoto (JP); Takeshi KUBO, Kyoto (JP); Takaaki OKANISHI, Kyoto (JP)

(21) Appl. No.: 19/453,547

(22) Filed: Jan. 20, 2026

Related U.S. Application Data
(63) Continuation of application No. PCT/JP2024/040416, filed on Nov. 14, 2024.

(30) Foreign Application Priority Data
Jan. 31, 2024 (JP) 2024-013758

(51) Int. Cl. A61B 5/0205; A61B 5/00; A61B 5/021; A61B 5/022; A61B 5/1455; A61B 5/256; A61B 5/282

(52) U.S. Cl. CPC A61B 5/0205; A61B 5/02141; A61B 5/02225; A61B 5/14552; A61B 5/256; A61B 5/282; A61B 5/681; A61B 2560/0462; A61B 2562/043

(57) ABSTRACT

A biological information measurement device configured to measure biological information, the device including: a plurality of electrodes; an electrode holding portion insulated from the electrodes, and having a contact surface that comes into contact with a surface of a measurement target during measurement of the biological information, the electrode holding portion being configured to hold at least one of the plurality of electrodes; and pressing means configured to press the electrodes against a skin surface of the measurement target at least during measurement of the biological information, in which the electrodes held by the electrode holding portion have a shape having a convex curved surface, and the curved surface is provided to project from the contact surface.

### CROSS-REFERENCE TO RELATED APPLICATIONS

This application is the U.S. national stage application filed pursuant to 35 U.S.C. 365(c) and 120 as a continuation of International Patent Application No. PCT/JP2024/040416, filed Nov. 14, 2024, which application claims priority to Japanese Patent Application No. 2024-013758, filed Jan. 31, 2024, which applications are incorporated herein by reference in their entireties.

### TECHNICAL FIELD

The present invention belongs to the technical field related to healthcare, and particularly relates to a biological information measurement device.

### BACKGROUND ART

It is known that a biological signal generated in a living body, such as an electrocardiographic signal, is measured using an electrode to be worn on a surface of the living body. In order to accurately measure the biological signal, it is necessary that the contact resistance between the electrode and the surface of the living body be sufficiently small. As a measure for that purpose, various configurations relating to the shape of the electrode have been conventionally known (for example, Patent Documents 1 and 2). Note that the electrodes described in Patent Documents 1 and 2 have a structure in which a protruding portion is provided on a flat plate, and the contact area between the flat plate portion as a part of the electrode and the surface of the living body varies depending on the contact method.

In addition, it has recently become common for an individual to measure information (hereinafter, also referred to as biological information) related to a body and health of the individual such as a blood pressure value and an electrocardiographic waveform on a daily basis by himself/herself by using a measurement apparatus and to utilize the measurement result for health management. Accordingly, there has been an increasing demand for apparatuses with an emphasis on portability, and many wearable measurement devices have become widespread.

### CITATION LIST

Patent Document 1: JP 2013-085629 A
Patent Document 2: JP 2016-036642 A
Patent Document 3: JP 2020-120915 A

### SUMMARY OF INVENTION

### Technical Problem

Meanwhile, in order to acquire an accurate measurement value, the contact resistance between the electrode and the surface of the living body needs to be sufficiently small, as described above, even in a device such as a wearable device that allows an individual to easily measure body information.

However, particularly in the case of using a simple measurement device such as a wearable terminal, even with the electrode structures described in Patent Documents 1 and 2, there is a problem that the contact area between the flat plate portion and the surface of the living body during the measurement of the biological signal significantly varies, and the acquired biological signal becomes unstable.

In view of the above-described problems, an object of the present invention is to provide a technique for reducing a variation in contact area between a surface of a living body and an electrode during measurement in a biological information measurement device including the electrode.

### Solution to Problem

A biological information measurement device according to the present invention adopts the following configurations in order to solve the above problems.

That is, a biological information measurement device configured to measure biological information, the device including: a plurality of electrodes; an electrode holding portion insulated from the electrodes and having a contact surface that comes into contact with a surface of a measurement target during measurement of the biological information, the electrode holding portion being configured to hold at least one of the plurality of electrodes; and pressing means configured to press the electrodes against a skin surface of the measurement target at least during measurement of the biological information, in which the electrodes held by the electrode holding portion have a shape having a convex curved surface, and the curved surface is provided to project from the contact surface.

According to such a configuration, the electrode having a structure projecting from the contact surface is pressed against the skin surface of the living body by the pressing means, and the electrode is buried in the human body so as to contact the peripheral surface of the projecting electrode, so that the entire electrode is easily maintained while being in contact with the living body. Furthermore, the electrode holding portion configured to hold the electrodes and forms the contact surface with the human body is insulated from the electrodes, and therefore the contact area between the contact surface and the skin surface does not affect the acquisition of a biological signal. Accordingly, it is possible to suppress a variation in the contact area between the electrode and the surface of the living body during measurement, and to acquire a stable signal (biological information).

In addition, each of the electrodes held by the electrode holding portion may be supported on a base end side of the electrode near the contact surface by a pedestal portion made of an insulator. According to such a configuration, a portion where the influence of body hairs on the skin surface increases (where the body hairs become concentrated when the electrode is pressed) may be made of an insulator, and a larger area of the electrode surface is likely to come into contact with the skin surface, thereby allowing a more stable signal to be acquired.

In addition, the biological information measurement device may be used with a main body case fixed to the measurement target by a band at least during measurement of the biological information, and the electrode holding portion may be provided on a side of the main body case on which the main body case comes into contact with the measurement target. Alternatively, a side of the band, on which the band comes into contact with the measurement target, may also be the electrode holding portion. In addition, the band may also be the pressing means. According to such a configuration, the present invention can be effectively applied to a wearable measurement device such as a wristwatch type.

In addition, the biological information measurement device may include the plurality of electrodes in the main body case, and may acquire an electrocardiographic signal on the basis of a potential difference between the plurality of electrodes. Alternatively, the biological information measurement device may include the plurality of electrodes on a side of the band on which the band comes into contact with the measurement target, and may acquire an electrocardiographic signal on the basis of a potential difference between the plurality of electrodes. In addition, the electrode holding portion may hold a measurement electrode used for measuring an electrocardiographic signal and a reference electrode for determining a reference potential.

In addition, the main body case may include an optical sensor between the measurement electrode and the reference electrode. In a multi-functional wearable biological information measurement device configured to measure other biological information such as a pulse wave (blood pressure), it is necessary to make the space where the electrodes are arranged compact. The present invention can be suitably used for such a configuration.

In addition, the biological information may include a blood pressure value, and the band may be provided with an air bag used for blood pressure measurement. In addition, the air bag may also be the pressing means.

In addition, the biological information measurement device may be a wearable device configured to enable the main body case to be worn on an arm portion of a human body that is the measurement target.

In addition, the electrodes held by the electrode holding portion may be formed to have any of a circular shape, an elliptical shape, or an oval shape in a plan view.

Note that the configurations and processing described above can be combined with one another to constitute the present invention unless the combination leads to technical contradiction.

### Advantageous Effects of Invention

According to the present invention, it is possible to provide a technique for reducing a variation in contact area between a surface of a living body and an electrode during measurement in a biological information measurement device including the electrode.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is an external perspective view illustrating an outline of a biological information measurement device according to a first embodiment of the present invention.

FIG. 2 is a side view illustrating the outline of the biological information measurement device according to the first embodiment.

FIG. 3 is an explanatory view illustrating an arrangement relationship when the biological information measurement device according to the first embodiment is worn a wrist.

FIG. 4 is an external view when a main body portion of the biological information measurement device according to the first embodiment is viewed from a bottom portion side.

FIG. 5 is a schematic cross-sectional view when the biological information measurement device according to the first embodiment is viewed from a side.

FIG. 6 is a schematic cross-sectional view of the vicinity of a sensor substrate housing portion of the biological information measurement device according to the first embodiment.

FIG. 7(A) is a schematic cross-sectional view for describing connection between electrodes and a sensor substrate of the biological information measurement device according to the first embodiment. FIG. 7(B) is an explanatory view for describing an electrode member according to the first embodiment. FIG. 7(C) is an explanatory view of a configuration of opening portions of a first sensor substrate according to the first embodiment.

FIG. 8 is a block diagram illustrating a functional configuration of the biological information measurement device according to the first embodiment.

FIG. 9(A) is a first explanatory view of a second modified example of the first embodiment. FIG. 9(B) is a second explanatory view of the second modified example of the first embodiment. FIG. 9(C) is a third explanatory view of the second modified example of the first embodiment.

FIG. 10(A) is a first explanatory view of a third modified example of the first embodiment. FIG. 10(B) is a second explanatory view of the third modified example of the first embodiment.

FIG. 11(A) is an external perspective view illustrating an outline of a biological information measurement device according to a second embodiment of the present invention. FIG. 11(B) is an explanatory view illustrating an outline of an inner circumferential surface of a belt portion of the second embodiment.

### DESCRIPTION OF EMBODIMENTS

### First Embodiment

Embodiments of the present invention will be specifically described below with reference to the drawings. Note that unless otherwise specified, the dimensions, materials, shapes, relative arrangements, and the like of the configurations described in the following embodiments are not intended to limit the scope of the present invention to those alone.

### Device Configuration

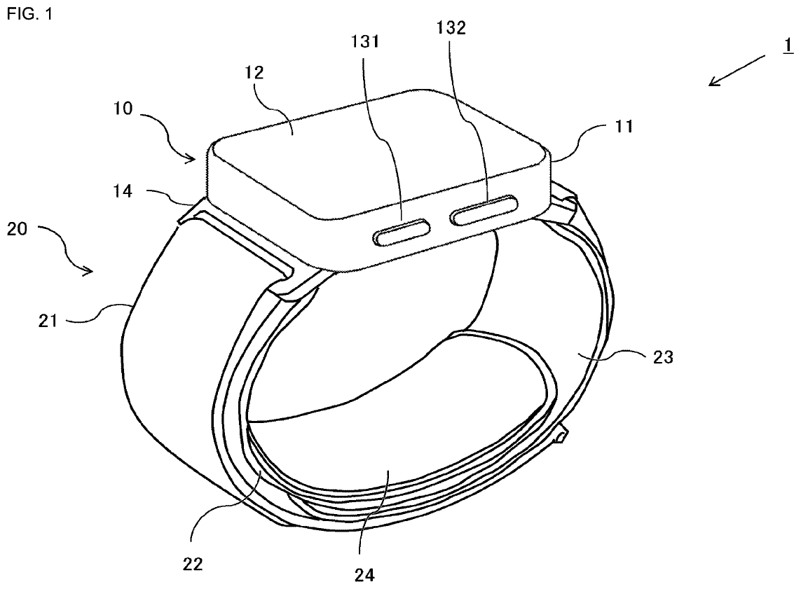

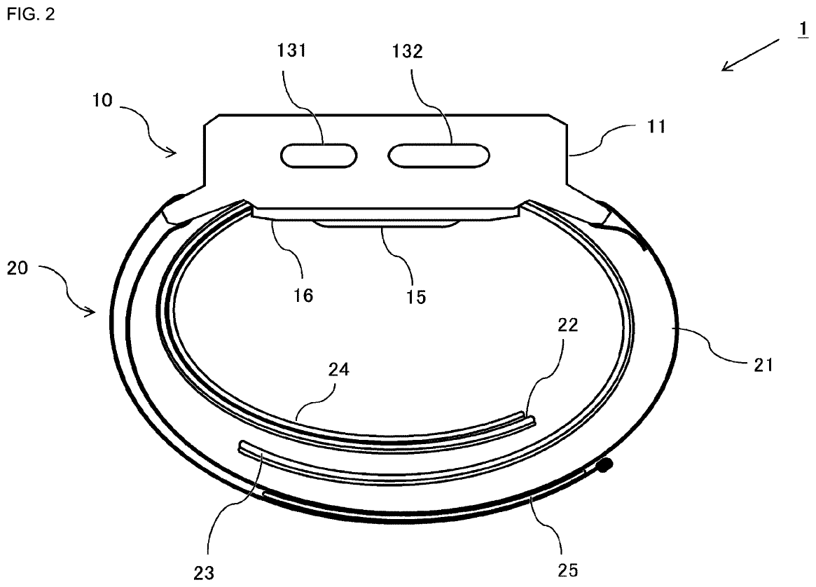

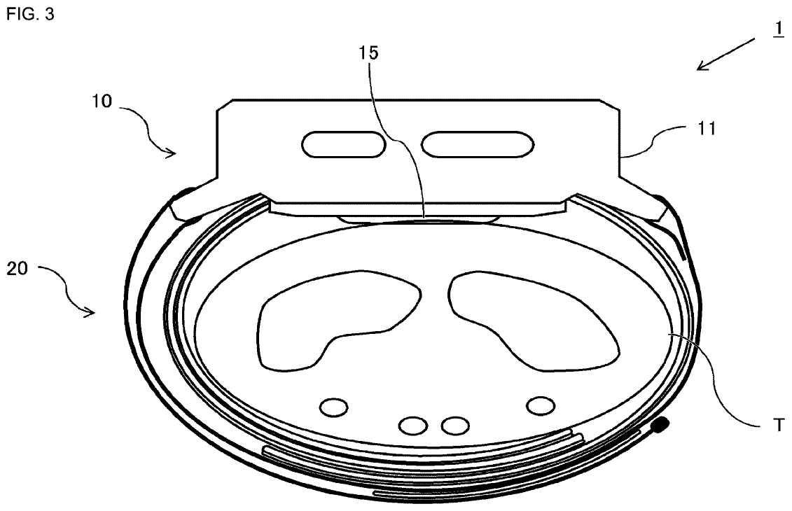

FIG. 1 is an external perspective view illustrating an outline of a configuration of a biological information measurement device 1 according to the present embodiment. In addition, FIG. 2 is a side view illustrating the outline of the configuration of the biological information measurement device 1 according to the present embodiment. As illustrated in FIG. 1 and FIG. 2, the biological information measurement device 1 is generally a wristwatch-type wearable device including a main body portion 10 and a belt portion 20, and is capable of measuring biological information such as a pulse wave (pulse), a blood pressure value, and an electrocardiographic waveform while being worn on a wrist T of a human body. FIG. 3 illustrates an arrangement relationship between the wrist T and configurations of the biological information measurement device 1 according to the present embodiment when the biological information measurement device 1 is worn on the wrist T.

As illustrated in FIG. 1 and FIG. 2, the main body portion 10 includes a main body case 11 and a cuff cover 16 described below. The main body case 11 is provided with a display 12 (for example, an organic EL display or the like), operation buttons 131 and 132, a lug 14, and the like, and is also provided with a sensor substrate housing portion 15 in which a sensor substrate is housed. Note that, in the present embodiment, a side on which the display 12 is formed is referred to as a front surface of the main body case 11, and a side on which the sensor substrate housing portion 15 is formed is referred to as a bottom portion of the main body case 11. In addition, in the following description, the front surface side of the main body case 11 may be referred to as the upper side, and the bottom portion side of the main body case 11 may be referred to as the lower side. Note that, in the present embodiment, the operation buttons 131 and 132 are formed of a conductor and also function as electrodes for electrocardiographic waveform measurement.

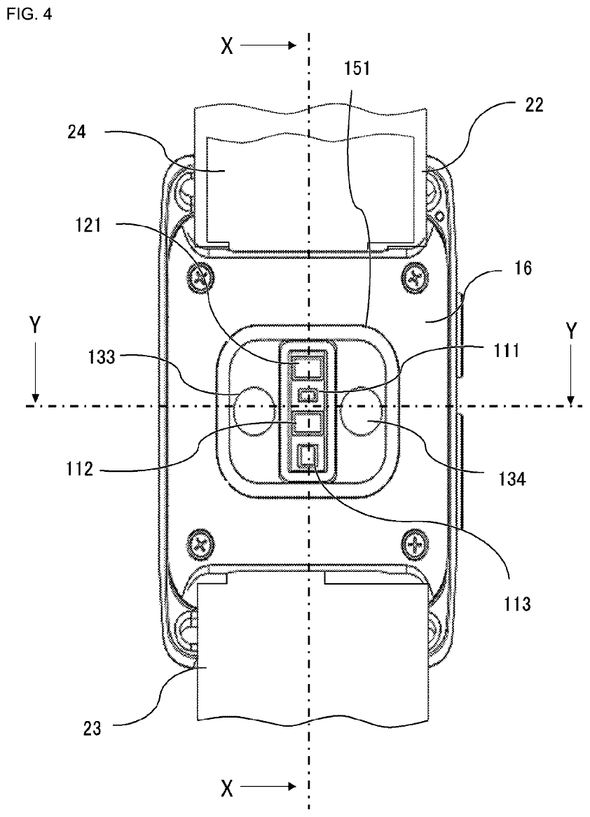

FIG. 4 illustrates an external view of the main body portion 10 when viewed from the bottom portion side. As illustrated in FIG. 4, the bottom portion of the main body case 11 includes a central region covered with a resin cover 151, and a region that corresponds to an outer periphery of the central region and is covered with the cuff cover 16. At least a portion of the resin cover 151 is formed of a light-transmissive resin, and an inner side of the main body case 11 in the region covered with the resin cover 151 corresponds to the sensor substrate housing portion 15. The sensor substrate housing portion 15 is located in the central region of the main body case 11 covered with the resin cover 151 in a plan view, and is formed so as to project toward the wrist T with respect to the cuff cover 16 in the worn state, as illustrated in FIG. 2 and FIG. 3. That is, a surface of the resin cover 151 on the bottom portion side serves as a contact surface that comes into contact with a human body.

In addition, a first electrode 133 and a second electrode 134 are provided at the bottom portion of the main body case 11 such that contact surfaces with the human body are exposed. One of the first electrode 133 and the second electrode 134 functions as a GND electrode during electrocardiographic waveform measurement. During the electrocardiographic waveform measurement, the biological information measurement device 1 is worn, the contact surfaces of the first electrode 133 and the second electrode 134 are brought into contact with the skin surface at the worn portion, and an operation button is touched with a finger on the side where the biological information measurement device 1 is not worn. Thus, the electrocardiographic waveform measurement can be performed by I induction. Note that the detailed structures of the first electrode 133 and the second electrode 134 will be described below.

In addition, although not illustrated, a charging terminal is also provided at the bottom portion of the main body case 11. A rechargeable battery (not illustrated in FIG. 4) can be charged by connecting a connection terminal of a power supply device and the charging terminal.

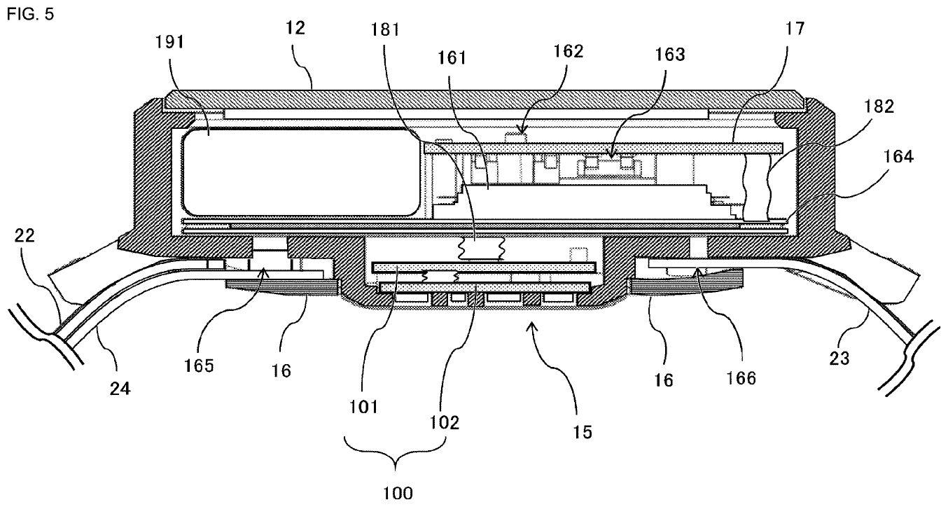

In addition, as illustrated in FIG. 5, a first LED 111, a second LED 113, a first photodiode (PD) 112, and a second PD 121 mounted on a lower surface (mounting surface) of a second sensor substrate 102 described below can be seen through the light-transmissive portion of the resin cover 151 from the bottom portion side of the main body case 11. These configurations will be described in detail below.

The belt portion 20 includes a belt 21 and a surface fastener 25 for fixing the biological information measurement device 1 to the wrist T, and also includes a first pressing cuff 22 and a second pressing cuff 23 for compressing an artery in the wrist T, and a sensing cuff 24 for detecting a pressure pulse wave. Note that connection portions between the respective cuffs 22, 23, and 24 and the main body case 11 are covered with the cuff cover 16. The cuff cover 16 protects the connection portions between the respective cuffs 22, 23, and 24 and the main body case 11, and also has a function to fix the respective cuffs 22, 23, and 24 to the main body case 11.

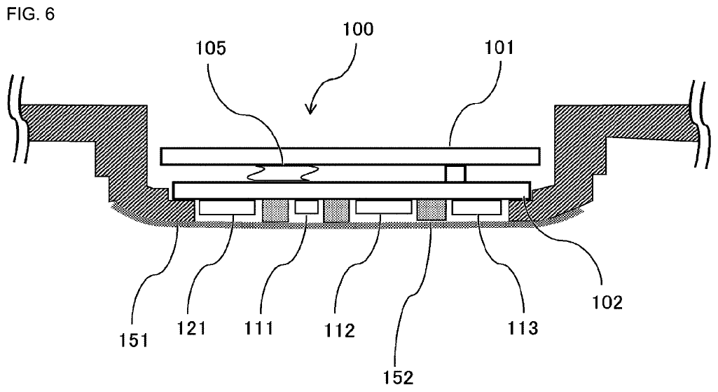

Next, the internal configuration of the main body case 11 will be described with reference to FIG. 5 and FIG. 6. FIG. 5 is a schematic cross-sectional view corresponding to an X-X cross-section of FIG. 4, and FIG. 6 is an enlarged view of the vicinity of the sensor substrate housing portion 15 in FIG. 5. Note that FIG. 5 and FIG. 6 are not accurate cross-sectional views, and the configuration is appropriately omitted and deformed for convenience of description. As illustrated in FIG. 5, a rechargeable battery 191, a control board 17, a piezoelectric pump 161, a valve 162, a pressure sensor 163, a flow path plate 164, and the like are housed inside the main body case 11. In addition, the sensor substrate housing portion 15 formed in a protrusion shape is provided in the vicinity of the bottom portion of the main body case 11, and a sensor substrate set 100 including a first sensor substrate 101 and a second sensor substrate 102 is housed in the sensor substrate housing portion 15.

The bottom portion of the main body case 11 is provided with a first connection portion 165 and a second connection portion 166 in a part of a region where the sensor substrate housing portion 15 is not provided in a plan view. The first connection portion 165 connects the main body case 11 (more specifically, the flow path plate 164 in the main body case) and the first pressing cuff 22 and the sensing cuff 24. The second connection portion 166 similarly connects the main body case 11 and the second pressing cuff 23. The first connection portion 165 and the second connection portion 166 are covered with the cuff cover 16 provided in the region corresponding to an outer periphery of the sensor substrate housing portion 15 on the bottom portion of the main body. In addition, as described above, the portion where the sensor substrate housing portion 15 is located is covered with the resin cover 151.

As the rechargeable battery 191, a general-purpose rechargeable battery such as a lithium ion battery can be adopted, and the rechargeable battery 191 can be repeatedly charged by receiving power supply via the charging terminal. In addition, a processor such as a CPU, a memory such as a RAM, and the like (not illustrated) are mounted on the control board 17, and the control board 17 performs entire control of the biological information measurement device 1. In addition, the piezoelectric pump 161, the valve 162, the pressure sensor 163, the flow path plate 164, the first pressing cuff 22, the second pressing cuff 23, and the sensing cuff 24 are configurations related to blood pressure measurement. The flow path plate 164 is a conductive member (metal), and a flow path for feeding gas from the piezoelectric pump 161 to each cuff is formed therein.

The first sensor substrate 101 and the flow path plate 164 are electrically connected by a spring contact 181, and the control board 17 and the flow path plate 164 are also electrically connected by a spring contact 182. Since the first sensor substrate 101 and the control board 17 are electrically connected to the conductive flow path plate 164, it is possible to increase the GND areas of the first sensor substrate 101 and the control board 17 and to improve noise resistance. In addition, for the first sensor substrate 101, the flow path plate 164 also functions as a shield against noise generated from the internal devices such as the piezoelectric pump 161.

Next, the sensor substrate housing portion 15 and the sensor substrate set 100 will be described. As illustrated in FIG. 6, the sensor substrate housing portion 15 is a space projecting from the bottom portion of the main body case 11 toward a side that comes into contact with the human body. The sensor substrate set 100, in which the first sensor substrate 101 and the second sensor substrate 102 are vertically stacked in two stages, is housed in the space. Note that the first sensor substrate 101 and the second sensor substrate 102 are connected by a conductive spring contact 105, and these two substrates function as a pair.

On a lower surface of the second sensor substrate 102, two light emitting elements of the first LED 111 and the second LED 113, and two light receiving elements of the first photodiode (PD) 112 and the second PD 121 are provided. Note that, in the present embodiment, the first LED 111 irradiates green irradiation light, and the second LED 113 irradiates red light and/or infrared light in addition to green light. Isolation walls 152 are also provided so as to isolate the first LED 111, the second LED 113, the first PD 112, and the second PD 121 from each other.

On the other hand, although not illustrated, a capacitor, an amplifier circuit, an analog-to-digital (A/D) conversion circuit, and the like are mounted on the first sensor substrate 101. Note that the first sensor substrate 101 may be a double-sided mounting substrate. In this way, the sensor substrate set 100 has a vertically stacked two-stage structure including the second sensor substrate 102 and the first sensor substrate 101. Thus, compared to a case where all components are mounted on one substrate, it is possible to significantly reduce the area of the substrates in a plan view.

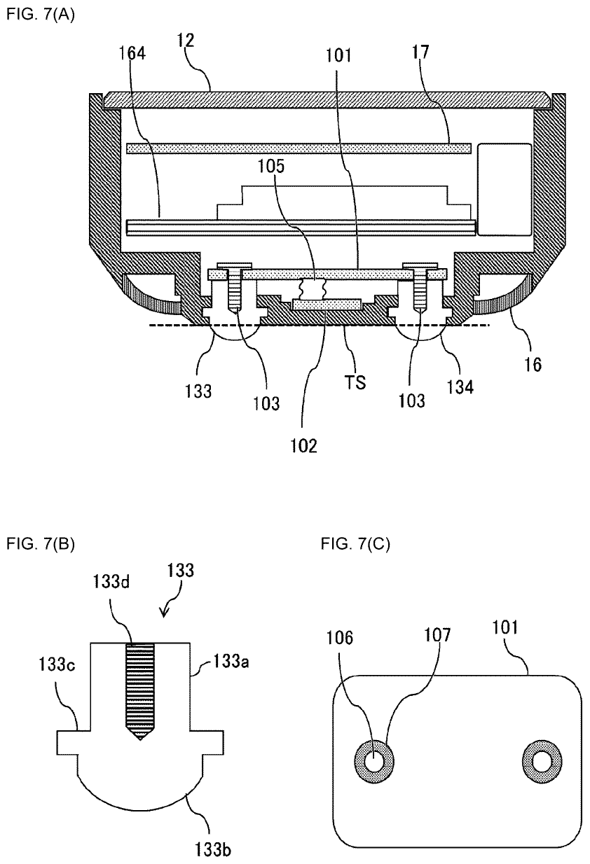

Next, a mode of connection between the first sensor substrate 101 and each electrode will be described with reference to FIG. 7(A), FIG. 7(B), and FIG. 7(C). FIG. 7(A) is a schematic cross-sectional view corresponding to a Y-Y cross-section of FIG. 4. However, FIG. 7(A) is not an accurate cross-sectional view, and the configuration is omitted and deformed for convenience of description. FIG. 7(B) is an explanatory view illustrating the structure of the first electrode 133. FIG. 7(C) is an explanatory view illustrating an outline of the lower surface of the first sensor substrate 101.

As illustrated in FIG. 7(A), the first electrode 133 and the second electrode 134 are fixed while being in contact with the lower surface of the first sensor substrate 101. In addition, both the electrodes are arranged such that a portion of each of the electrodes projects from a contact surface TS (a surface located on a line indicated by a broken line in FIG. 7(A)), which is a surface on the bottom portion side of the resin cover 151, toward a side that comes into contact with the human body when the device is worn.

Here, the first electrode 133 and the second electrode having the same configuration will be described in more detail with reference to FIG. 7(B). The first electrode 133 is substantially constituted by a shaft portion 133a having a longitudinal direction in the vertical direction of the drawing sheet and a head portion 133b having a curved surface projecting from the contact surface TS of the resin cover 151 with the human body. Note that the head portion 133b is circular in a plan view and has a so-called dome shape. Note that a height of the projection portion of the head portion 133b from the contact surface TS, a diameter of the projection portion in a plan view, and the like are not particularly limited, but may, for example, fall within a height range of 1 mm to 3 mm, a diameter range of 3 mm to 7 mm, and the like.

The shaft portion 133a is formed in a hollow cylindrical shape, and an inner wall thereof is provided with a screw portion 133d. That is, the shaft portion 133a functions as a female screw. In addition, a flange-shaped retaining protrusion portion 133c is formed on a lower side of the shaft portion 133a. The retaining protrusion portion 133c is fixed in engagement with a recess portion provided in an inner wall on the bottom portion side of the main body case 11, and thus the first electrode 133 is held by the main body case 11. For example, such a structure can be realized by insert-molding the first electrode 133 into the main body case 11. Note that although the first electrode 133 has been described here, the same also applies to the second electrode 134. In the present embodiment, the main body case 11 (the bottom side thereof) corresponds to the holding portion in the present invention.

As illustrated in FIG. 7(C), opening portions 106 are provided in the first sensor substrate 101, and an electrode pad 107 is formed on an outer periphery of each opening portion. As illustrated in FIG. 7(A), the first electrode 133 and the second electrode 134 are screwed with screw members 103 serving as male screws via the opening portions 106 of the first sensor substrate 101, and are thereby fixed to the first sensor substrate 101. The fixing is performed in a state in which the distal end surfaces of the shaft portions of the first electrode 133 and the second electrode 134 are in contact with the electrode pads 107 formed at the outer peripheries of the opening portions 106 of the first sensor substrate 101. Thus, the first electrode 133 and the second electrode 134 are fixed to the first sensor substrate 101 while being electrically connected to the first sensor substrate 101.

### Functional Configuration of Device

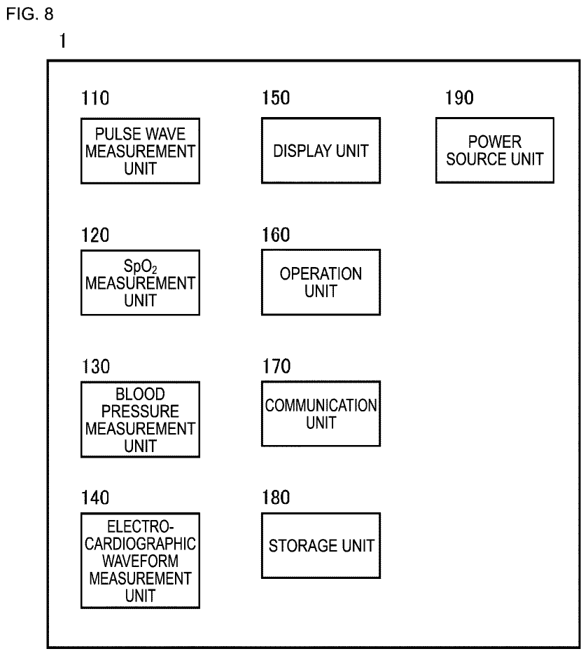

Next, a functional configuration of the biological information measurement device 1 will be described. FIG. 8 is a block diagram illustrating the functional configuration of the biological information measurement device 1. As illustrated in FIG. 8, the biological information measurement device 1 according to the present embodiment includes functional units of a pulse wave measurement unit 110, a blood oxygen saturation (SpO₂) measurement unit 120, a blood pressure measurement unit 130, an electrocardiographic waveform measurement unit 140, a display unit 150, an operation unit 160, a communication unit 170, a storage unit 180, and a power source unit 190. The processor of the control board 17 reads a program from the memory and executes the program to control each configuration of the biological information measurement device 1, thereby implementing these functional units.

The pulse wave measurement unit 110 includes the first LED 111, the second LED 113, and the first PD 112, and measures a pulse wave by a so-called photoplethysmographic method to calculate a pulse. Specifically, green light is irradiated from the first LED 111 and the second LED 113, and reflected light reflected in the living body is received by the first PD 112, so that a blood flow volume that changes with a pulsation of the heart (a change in volume of a blood vessel) is detected, and a pulse wave is measured.

The SPO₂ measurement unit 120 includes the second LED 113 and the second PD 121, and receives reflected light of red light or infrared light irradiated from the second LED 113 with the second PD 121 to measure a blood oxygen saturation from an intensity of the reflected light.

The blood pressure measurement unit 130 includes the piezoelectric pump 161, the valve 162, the pressure sensor 163, the flow path plate 164, the first pressing cuff 22, the second pressing cuff 23, and the sensing cuff 24, and measures a blood pressure by a so-called oscillometric method. The blood pressure measurement using the oscillometric method is a known technique, and therefore the detailed description thereof is omitted.

The electrocardiographic waveform measurement unit 140 includes the operation buttons 131 and 132, the first electrode 133 and the second electrode 134 provided at the bottom portion of the main body case 11, and an electrocardiographic waveform measuring circuit (not illustrated), and measures an electrocardiographic waveform by a so-called I-induction method. Specifically, an electrocardiographic waveform is measured based on a potential difference between the first electrode 133 and the second electrode 134 that are in contact with the wrist T of one arm in the worn state, and a finger of the other hand in contact with the operation button 131 or 132 functioning as an electrode.

The display unit 150 includes the display 12, and displays various types of information such as a measurement result of biological information and a menu screen. The operation unit 160 includes the operation buttons 131 and 132, and receives an input operation by the user via these buttons. The communication unit 170 includes an antenna for wireless communication (not illustrated), and performs information communication with another electronic device such as an information processing terminal by, for example, BLE communication. Note that a terminal for wired communication may be provided.

The storage unit 180 includes a main storage device (not illustrated) such as a random access memory (RAM) and stores various types of information such as application programs and measured biological information. In addition, it may include a long-term storage medium such as a flash memory in addition to the RAM, for example. The power source unit 190 includes the rechargeable battery 191 and the charging terminal 192, and functions as a power supply source to each unit constituting the biological information measurement device 1.

Measurement of Biological Information and Effects of Present Embodiment According to the biological information measurement device 1 described above, the blood pressure and the electrocardiographic waveform can be measured at the same time. At this time, the fluid flows into the first pressing cuff 22 and the second pressing cuff 23, and thus the wrist T and the bottom surface side of the main body case 11 are pressed against each other. Therefore, the head portions of the first electrode 133 and the second electrode 134 projecting from the contact surface TS of the main body case 11 are buried in the skin surface of the human body. That is, in the present embodiment, the first pressing cuff 22 and the second pressing cuff 23 are configured to also serve as the pressing means according to the present invention.

With such a configuration, the entire head portion of each of the first electrode 133 and the second electrode 134 is easily maintained while being in contact with the living body. Furthermore, the bottom surface side of the main body case 11 that holds the first electrode 133 and the second electrode 134 and forms the contact surface TS with the human body is the resin cover 151, and is insulated from each electrode. Therefore, even if the contact area between the contact surface TS and the skin surface varies during the measurement of the electrocardiographic waveform, the acquired electrocardiographic signal is not affected. Accordingly, it is possible to suppress a variation in the contact area between the first electrode 133 and second electrode 134 and the skin surface during measurement, and to acquire a stable signal.

Note that even when only the electrocardiographic waveform is measured (that is, even when there is no pressing by the first pressing cuff 22 and the second pressing cuff 23), the head portions of the first electrode 133 and the second electrode 134 are fixed while being buried in the skin surface of the human body as long as the main body case 11 is firmly fixed and worn on the wrist T by the belt 21 and the surface fastener 25. Therefore, the blood pressure measurement and the electrocardiogram measurement do not necessarily need to be performed at the same time. Note that, in this case, the tightening force of the belt portion 20 also acts as the pressing force of the first electrode 133 and the second electrode 134 against the skin surface, and thus the belt portion 20 corresponds to the pressing means in the present invention.

### First Modified Example

Note that the shapes of the first electrode 133 and the second electrode 134 are not particularly limited as long as they have a structure having a curved surface projecting from the contact surface TS, and various shapes can be adopted. For example, the first electrode 133 (and the second electrode 134) may not include the retaining protrusion portion 133c. In this case, it is not necessary to provide the retaining recess portion in the bottom portion of the main body case 11, and the surface on the proximal end side of the head portion and the main body case 11 may be bonded and fixed to each other with an adhesive, for example.

### Second Modified Example

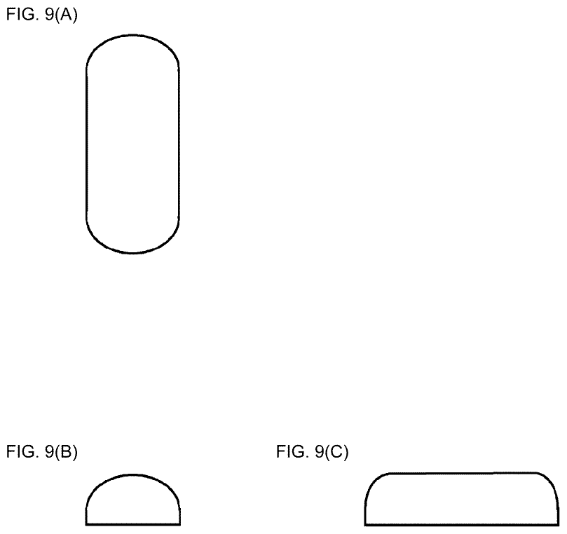

In addition, the head portions of the first electrode 133 and the second electrode 134 may have an elliptical shape or an oval shape in a plan view, instead of a circular shape. FIGS. 9(A) to 9(C) are diagrams illustrating shapes of an electrode having an oval shape in a plan view as an example of such a modified example. FIG. 9(A) is a schematic plan view of an electrode according to a modified example, FIG. 9(B) is a schematic view illustrating a side surface in a lateral direction of a head portion (that is, a projection portion from the contact surface TS) of the electrode according to the modified example, and FIG. 9(C) is a schematic view illustrating a side surface in a longitudinal direction of the head portion of the electrode according to the modified example. In addition, the head portion of the electrode may also have a shape close to a rounded square in a plan view as long as the head portion is formed to have a curved surface.

### Third Modified Example

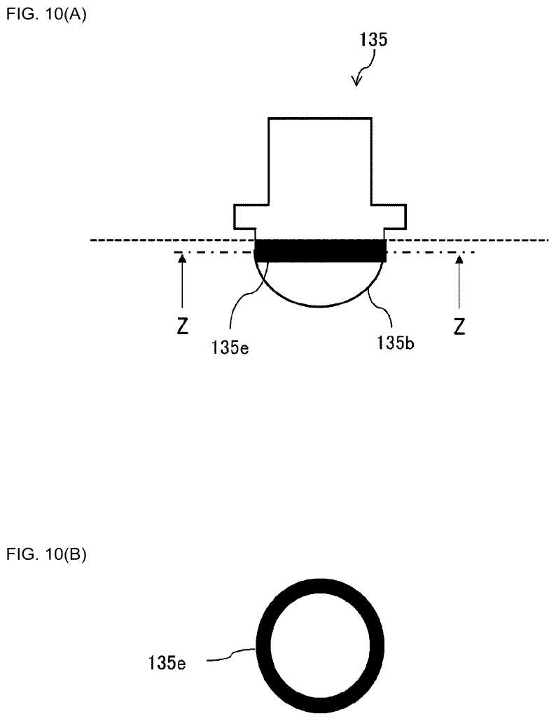

In addition, the first electrode 133 and the second electrode 134 may be configured such that the proximal end side of the head portion is made of an insulator. FIG. 10(A) and FIG. 10(B) illustrate an explanatory view of such a modified example. FIG. 10(A) is a schematic side view of a first electrode 135 according to the modified example, and FIG. 10(B) is a schematic cross-sectional view corresponding to a Z-Z cross section of FIG. 10(A). Note that the broken line in FIG. 10(A) indicates a line on which the contact surface TS of the main body case 11 is located. As illustrated in FIG. 10(A) and FIG. 10(B), in the first electrode 135 according to the present modified example, a portion from an outer periphery toward a center on a proximal end side of a head portion 135b projecting from the contact surface is a pedestal portion 135e made of resin (i.e., an insulator).

When the head portion 135b is pressed against the skin surface, the body hairs on the skin surface are concentrated on the proximal end side of the head portion 135b. Although an increase in the contact area between the electrode and the body hairs adversely affects the acquisition of stable biological signals, such an adverse effect can be reduced by forming the proximal end side (the outer periphery thereof) of the head portion 135b, where the body hairs are concentrated, to be made of an insulator.

### Second Embodiment

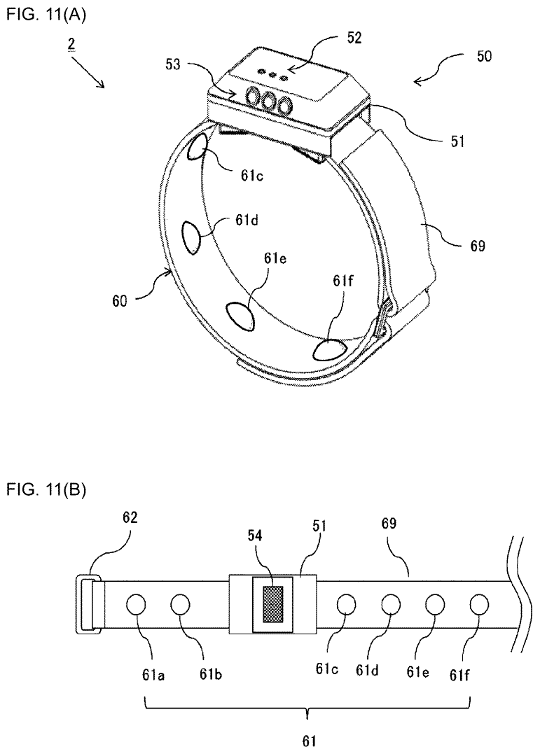

Next, another embodiment of the present invention will be described based on FIGS. 11(A) and 11(B). FIG. 11(A) and FIG. 11(B) are schematic views illustrating a configuration of a biological information measurement device 2 in a second embodiment, in which FIG. 11(A) illustrates an external perspective view of the biological information measurement device 2, and FIG. 11(B) illustrates an outline of an inner circumferential surface of a belt portion 60 of the biological information measurement device 2.

As illustrated in FIG. 11(A) and FIG. 11(B), the biological information measurement device 2 generally includes a main body portion 50 including a main body case 51, a control unit (not illustrated), an LED indicator 52, an operation button 53, a pulse wave sensor 54, and the like, and a belt portion 60 including a belt 69 made of a resin, an electrode portion 61 composed of a plurality of electrodes 61a, 61b, 61c, 61d, 61e, and 61f, and a belt loop 62.

Note that although not illustrated, the belt 69 is provided with a surface fastener portion including hooks and loops. A user can wear the biological information measurement device 2 by arranging the biological information measurement device 2 on, for example, a left upper arm portion such that the respective electrodes come into contact with the skin surface, and inserting one end portion of the belt 69 through the belt loop 62, folding back the one end portion, and engaging the hook-and-loop fastener to fix the belt 69 in a ring shape to the upper arm.

The electrode portion 61 includes six electrodes 61a, 61b, 61c, 61d, 61e, and 61f, and the respective electrodes are electrically connected to the main body portion 50 via electrical lines (not shown) or the like arranged in the belt portion 60. Accordingly, the electrode portion 61 functions as a sensor unit that detects an electrocardiographic signal. Specifically, in a state in which the biological information measurement device 2 is worn, two of the electrodes in an opposing positional relationship pair up with each other, and an electrocardiographic signal is detected based on a potential difference between the two electrodes in a pair. In other words, three types of electrocardiographic signals can be simultaneously detected from three pairs of electrodes.

As illustrated in FIGS. 11(A) and 11(B), each of the electrodes 61a, 61b, 61c, 61d, 61e, and 61f is circular when viewed from an inner side of the belt 69 made of resin (a side that comes into contact with the skin surface), and is configured to project from an inner surface of the belt 69 in a dome shape. When the biological information measurement device 2 is worn, the respective electrodes are pressed against the skin surface by the tightening force of the belt 69, and are thereby buried in and fixed to the skin surface. That is, in the present embodiment, the belt 69 corresponds to the electrode holding portion and the pressing means according to the present invention.

Note that the pulse wave sensor 54 functions as a sensor unit that detects a pulse wave signal. The pulse wave sensor 54 in the present embodiment is a reflection type photoelectric pulse wave sensor arranged on a lower surface side of the main body case 51 (i.e., a surface that comes into contact with the skin when worn) as illustrated in FIG. 11(B). The reflection type photoelectric pulse wave sensor can detect a blood flow volume (a change in volume of a blood vessel) that changes with a pulsation of the heart by irradiating a living body with infrared light, red light, or green light and detecting, with the use of a photodiode or the like, the light reflected in the living body. In addition, based on this, it is possible to further measure (estimate) a blood pressure value or the like.

### Supplemental Note

The descriptions of the above examples are merely illustrative of the present invention, and the present invention is not limited to the specific embodiments described above. Various modified examples and combinations may be made within the scope of the technical idea of the present invention. For example, the biological information measurement device is only required to include an electrode and a circuit for measuring an electrocardiographic waveform, and a function and a configuration for acquiring other biological information are not necessarily indispensable.

In addition, in the above examples, the biological information measurement device has a configuration in which the main body case is fixed to the living body by the belt (band), that is, the measurement device main body and the electrode holding portion are integrated, but the present invention can also be applied to other biological information measurement devices. Specifically, the present invention can be applied to a biological information measurement device having a configuration in which an electrode is provided in a probe extending from a stationary main body, for example.

In addition, the pressing means may simply have a function of relatively pressing the electrode against the skin surface, and means such as a suction cup that draws the skin surface toward the electrode by suction to cause the projection portion of the electrode to be buried in the skin surface may be adopted. In addition, the contact surface of the electrode holding portion from which the electrode projects may be attached to the skin surface with an adhesive, thereby causing the projection portion of the electrode to be buried in the skin surface. In this case, the contact surface (electrode holding portion) to which the adhesive is applied serves as the pressing means.

In addition, various modifications can be made to the shapes of the respective electrodes in the second embodiment in the same manner as described in the first embodiment.

### REFERENCE SIGNS LIST

1,2 . . . Biological information measurement device
10, 50 . . . Main body portion
11,51 . . . Main body case
12 . . . Display
14 . . . Lug
15 . . . Sensor substrate housing portion
16 . . . Cuff cover
17 . . . Control board
20, 60 . . . Belt portion
21, 69 . . . Belt
22 . . . First pressing cuff
23 . . . Second pressing cuff
24 . . . Sensing cuff
25 . . . Surface fastener
52 . . . LED indicator
53 . . . Operation button
54 . . . Pulse wave sensor
61a, 61b, 61c, 61d, 61e, 61f . . . Electrode
62 . . . Belt loop
100 . . . Sensor substrate set
101 . . . First sensor substrate
102 . . . Second sensor substrate
103 . . . Screw member
105 . . . Spring contact
106 . . . Opening portion
107 . . . Electrode pad
111 . . . First LED
112 . . . First PD
113 . . . Second LED
121 . . . Second PD
131, 132 . . . Operation button
133, 135 . . . First electrode
134 . . . Second electrode
151 . . . Resin cover
152 . . . Isolation wall
161 . . . Piezoelectric pump
162 . . . Valve
163 . . . Pressure sensor
164 . . . Flow path plate
165 . . . First connection portion
166 . . . Second connection portion
191 . . . Rechargeable battery
T . . . Wrist
TS . . . Contact surface

What is claimed is:

1. A biological information measurement device configured to measure biological information, the device comprising:
a plurality of electrodes;
an electrode holding portion insulated from the electrodes and having a contact surface that comes into contact with a surface of a measurement target during measurement of the biological information, the electrode holding portion being configured to hold at least one of the plurality of electrodes; and,
pressing means configured to press the electrodes against a skin surface of the measurement target at least during measurement of the biological information,
wherein the electrodes held by the electrode holding portion have a shape having a convex curved surface, and the curved surface is provided to project from the contact surface,
each of the electrodes includes a base end portion projecting from the contact surface and having a smaller outer diameter than a base end of the convex curved surface on a side closer to the contact surface relative to the curved surface; and,
each of the electrodes includes a pedestal portion made of an insulator on an outer periphery of the base end portion.

2. The biological information measurement device according to claim 1, wherein:
the biological information measurement device is used with a main body case fixed to the measurement target by a band at least during measurement of the biological information; and,
the electrode holding portion is provided on a side of the main body case on which the main body case comes into contact with the measurement target.

3. The biological information measurement device according to claim 2, wherein the biological information measurement device comprises the plurality of electrodes in the main body case, and acquires an electrocardiographic signal on a basis of a potential difference between the plurality of electrodes.

4. The biological information measurement device according to claim 3, wherein the electrode holding portion holds a measurement electrode used for measuring an electrocardiographic signal and a reference electrode for determining a reference potential.

5. The biological information measurement device according to claim 4, wherein the main body case includes an optical sensor between the measurement electrode and the reference electrode.

6. The biological information measurement device according to claim 2, wherein:
the biological information includes a blood pressure value; and,
the band is provided with an air bag used for blood pressure measurement.

7. The biological information measurement device according to claim 1, wherein:
the biological information measurement device is used with a main body case fixed to the measurement target by a band at least during measurement of the biological information; and,
a side of the band, on which the band comes into contact with the measurement target, is the electrode holding portion.

8. The biological information measurement device according to claim 7, wherein the biological information measurement device comprises the plurality of electrodes in the electrode holding portion, and acquires an electrocardiographic signal on a basis of a potential difference between the plurality of electrodes.

9. The biological information measurement device according to claim 7, wherein:
the biological information includes a blood pressure value; and,
the band is provided with an air bag used for blood pressure measurement.

10. The biological information measurement device according to claim 2, wherein the biological information measurement device is a wearable device configured to enable the main body case to be worn on an arm portion of a human body that is the measurement target.

11. The biological information measurement device according to claim 10, wherein the band is the pressing means.

12. The biological information measurement device according to claim 6, wherein the air bag is the pressing means.

13. The biological information measurement device according to claim 9, wherein the air bag is the pressing means.

14. The biological information measurement device according to claim 1, wherein the electrodes held by the electrode holding portion are formed to have any of a circular shape, an elliptical shape, or an oval shape in a plan view.
